(19) United States
(12) Patent Application Publication
(10) Pub. No.: US 2026/0060659 A1
(43) Pub. Date: Mar. 5, 2026

(54) METHOD AND SYSTEM FOR AUTOMATIC EXECUTION OF PRE-DEFINED ACTIONS BASED ON ANATOMICAL STRUCTURE SEGMENTATION AND SELECTION DURING ULTRASOUND IMAGING

(71) Applicant: GE Precision Healthcare LLC, Waukesha, WI (US)

(72) Inventors: Doron Shaked, Tivon (IL); Eran Shahar, Haifa (IL); Yelena Tsymbalenko, Mequon, WI (US); Michael Ledwidge, Waunakee, WI (US); Michael Washburn, Brookfield, WI (US); Shunichiro Tanigawa, Toyko (JP); Amitay Lev, Or Akiva (IL)

(21) Appl. No.: 18/825,768

(22) Filed: Sep. 5, 2024

(51) Int. Cl. A61B 8/00

(52) U.S. Cl. CPC A61B 8/54; A61B 8/463; A61B 8/469

(57) ABSTRACT

A system and method for automatically executing pre-defined actions based on anatomical structure segmentation and selection during ultrasound imaging is provided. The method includes acquiring, by an ultrasound probe of an ultrasound system, first ultrasound image information according to a first mode. The first ultrasound image information includes a first mode ultrasound image depicting anatomical structures. The method includes processing, by at least one processor of the ultrasound system, the first mode ultrasound image to segment each of the anatomical structures depicted in the first mode ultrasound image. The method includes causing a display system to present the first mode ultrasound image. The method includes receiving a user interaction selecting one of the anatomical structures segmented in the first mode ultrasound image. The method includes triggering, in response to the user interaction, execution of a pre-defined action associated with the one of the anatomical structures selected by the user interaction.

### FIELD

Certain embodiments relate to ultrasound imaging. More specifically, certain embodiments relate to a method and system for automatically executing pre-defined action associated with selected anatomical structures segmented in an acquired ultrasound image.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Standard ultrasound imaging views of an abdomen typically include multiple patient anatomical structures, such as a liver, kidney, gallbladder, aorta, pancreas, spleen, and/or inferior vena cava, for example. If an ultrasound operator initiates a Color Flow mode, Power Doppler mode, B-Flow Color mode, or the like, selecting an appropriate present of ultrasound imaging settings for the particular anatomical structure is important for image quality. Manually setting an appropriate preset may be inefficient and cumbersome. Automating the selection of the appropriate setting is also difficult due to the inherent view ambivalence. Specifically, in many cases, an acquired ultrasound image may qualify as a valid view for more than one anatomical structure. Accordingly, although an ultrasound operator knows a target anatomical structure when initiating a Color Flow mode, Power Doppler mode, B-Flow Color mode, or the like, automatically setting a correct preset may not be possible in situations where multiple anatomical structures are depicted, and manually setting a correct preset is inefficient and cumbersome. Similar problems are present when attempting to automate a variety of pre-defined tasks associated with particular anatomical structures when multiple anatomical structures are depicted in an ultrasound image, such as optimized imaging settings for ultrasound image acquisition of a particular anatomical structure, selection of a standard labeling form associated with a particular anatomical structure, selection of a measurement associated with a particular anatomical structure, and the like.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for automatically executing pre-defined actions based on anatomical structure segmentation and selection during ultrasound imaging, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to automatically execute pre-defined actions based on anatomical structure segmentation and selection during ultrasound imaging, in accordance with various embodiments.

FIG. 2 illustrates screenshots of an exemplary first mode ultrasound image and an exemplary first mode ultrasound image having identifications of segmented anatomical structures, in accordance with various embodiments.

FIG. 3 illustrates a screenshot of exemplary a region of interest box placed on a first mode ultrasound image, in accordance with various embodiments.

FIG. 4 illustrates a screenshot of exemplary second mode ultrasound information within a region of interest box placed on a first mode ultrasound image, in accordance with various embodiments.

FIG. 5 illustrates a screenshot of another exemplary second mode ultrasound information within a region of interest box placed on a first mode ultrasound image, in accordance with various embodiments.

FIG. 6 illustrates a screenshot of another exemplary second mode ultrasound information within a region of interest box placed on a first mode ultrasound image, in accordance with various embodiments.

FIG. 7 illustrates a screenshot of another exemplary second mode ultrasound information within a region of interest box placed on a first mode ultrasound image, in accordance with various embodiments.

FIG. 8 illustrates a screenshot of an exemplary first mode ultrasound image having identifications of segmented anatomical structures presented with a pictogram having representations of anatomical structures corresponding with the segmented anatomical structures in the first mode ultrasound image, in accordance with various embodiments.

FIG. 9 illustrates a screenshot of exemplary measurement in a first mode ultrasound image, in accordance with various embodiments.

FIG. 10 illustrates a screenshot of an exemplary first mode ultrasound image having a set of labels corresponding with a selected anatomical structure, in accordance with various embodiments.

FIG. 11 is a flow chart illustrating exemplary steps that may be utilized for automatically executing pre-defined actions based on anatomical structure segmentation and selection during ultrasound imaging, in accordance with various embodiments.

### DETAILED DESCRIPTION

Certain embodiments may be found in a method and system for automatically executing pre-defined actions based on anatomical structure segmentation and selection during ultrasound imaging. For example, aspects of the present disclosure have the technical effect of automatically segmenting a plurality of anatomical structures in a first mode ultrasound image and receiving a selection of a target anatomical structure associated with one or more pre-defined actions in order to automatically execute the pre-defined actions. As an example, the pre-defined actions may include automatically selecting imaging settings specific to acquisition of additional ultrasound images of a target anatomical structure according to the first mode. As another example, the pre-defined actions may include automatically selecting imaging settings specific to acquisition of ultrasound information of a target anatomical structure acquired according to a second mode, different from the first mode. As another example, the pre-defined actions may include automatically performing a measurement of a selected target anatomical structure. As another example, the pre-defined actions may include automatically labeling the first mode ultrasound image according to a standard labeling form associated with the selected target anatomical structure. The selection of the target anatomical structure may be a selection of an identification of one of a plurality of anatomical structures segmented in the first mode ultrasound image. The selection of the target anatomical structure may be a placement of a region of interest box associated with a second ultrasound imaging mode. The selection of the target anatomical structure may be a selection of an anatomical structure in a pictogram having a plurality of anatomical structures corresponding with the segmented plurality of anatomical structures in the first mode ultrasound image. In various embodiments, the plurality of anatomical structures in the pictogram may be color-coded, labeled, or otherwise represented in a manner that is the same as the identifications of the plurality of anatomical structures segmented in the first mode ultrasound image.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode or 2D mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-Flow Color (BFC), Micro Vascular Imaging (MVI), Ultrasound-Guided Attenuation Parameter (UGAP), and the like.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

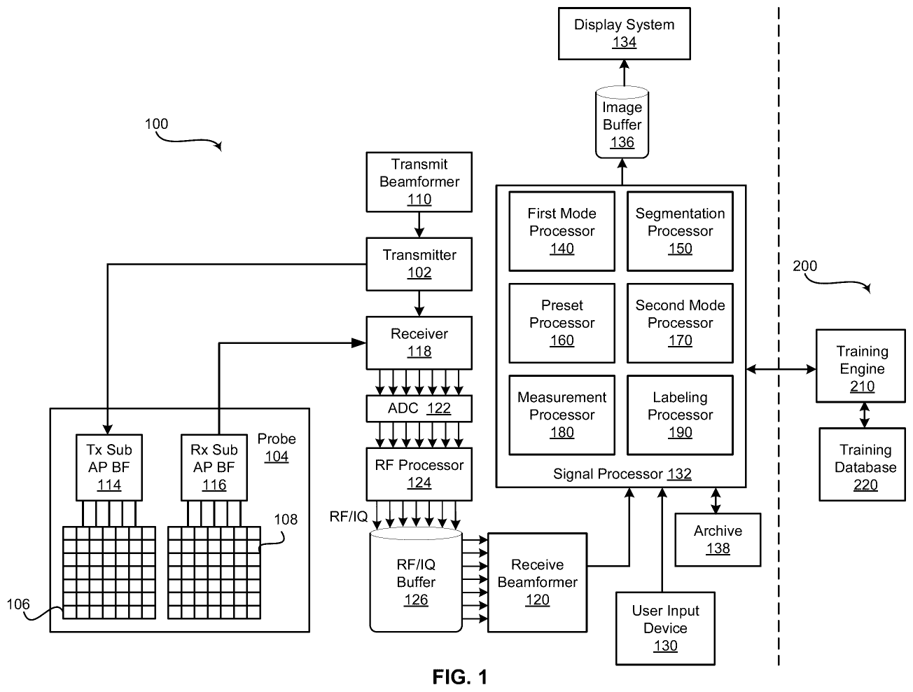

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to automatically execute pre-defined actions based on anatomical structure segmentation and selection during ultrasound imaging, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a radio frequency (RF) processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two-dimensional (2D) array of piezoelectric elements. In various embodiments, the ultrasound probe 104 may be a matrix array transducer or any suitable transducer operable to acquire 2D and/or 3D (including 4D) ultrasound image datasets. The ultrasound probe 104 may comprise a group of transmit transducer elements 106 and a group of receive transducer elements 108, that normally constitute the same elements. In certain embodiments, the ultrasound probe 104 may be operable to acquire ultrasound image data covering at least a substantial portion of an anatomy, such as an abdomen, a heart, a fetus, a lung, a blood vessel, or any suitable anatomical structure(s).

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be backscattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or a plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form in-phase and quadrature (IQ) data pairs that are representative of the corresponding echo signals. The RF or IQ signal data may then be communicated to an RF/IQ buffer 126. The RF/IQ buffer 126 may comprise suitable logic, circuitry, interfaces and/or code that may be operable to provide temporary storage of the RF or IQ signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, image acquisition and scan parameters, settings, configuration parameters, select protocols and/or templates, change scan mode, select anatomical structure targets, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise a first mode processor 140, a segmentation processor 150, a preset processor 160, a second mode processor 170, a measurement processor 180, and a labeling processor 190 that may be capable of receiving input information from a user input device 130 and/or archive 138, generating an output displayable by a display system 134, and manipulating the output in response to input information from a user input device 130, among other things. The signal processor 132, first mode processor 140, segmentation processor 150, preset processor 160, second mode processor 170, measurement processor 180, and labeling processor 190 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a first mode processor 140 that comprises suitable logic, circuitry, interfaces and/or code that may be operable to process an acquired and/or retrieved first mode ultrasound image dataset to generate ultrasound images according to a first mode. As an example, the first mode may be a 2D mode (e.g., B-mode, biplane mode, triplane mode, or the like) and the first mode processor 140 may be configured to process a received first mode ultrasound image dataset into 2D image(s). The first mode image(s) may be provided to the segmentation processor 150, presented at the display system 134, and/or stored at archive 138 or any suitable data storage medium.

The signal processor 132 may include a segmentation processor 150 that comprises suitable logic, circuitry, interfaces and/or code that may be operable to process the first mode ultrasound image generated by the first mode processor 140 to segment a plurality of anatomical structures depicted in the first mode ultrasound image. For example, the segmentation processor 150 may determine locations and boundaries of one or more of a liver, kidney, gallbladder, aorta, pancreas, spleen, and/or inferior vena cava, among other anatomical structures, depicted in a first mode ultrasound image of an abdomen. In various embodiments, the segmentation processor 150 may receive a first mode ultrasound image from the first mode processor 140 and/or may retrieve the first mode ultrasound image from archive 138 and/or any suitable data storage medium.

The segmentation processor 150 may include image analysis algorithms, artificial intelligence algorithms, one or more deep neural networks (e.g., a convolutional neural network) and/or may utilize any suitable form of image analysis techniques or machine learning processing functionality configured to automatically determine locations and boundaries of anatomical structures depicted in a first mode ultrasound image. In various embodiments, the segmentation processor 150 may be provided as a deep neural network that may be made up of, for example, an input layer, an output layer, and one or more hidden layers in between the input and output layers. In an exemplary embodiment, the deep neural network may be an object segmentation model that identifies boundaries of anatomical structures on a pixel-by-pixel basis within the first mode ultrasound image. Each of the layers may be made up of a plurality of processing nodes that may be referred to as neurons. For example, the segmentation processor 150 may include an input layer having a neuron for each pixel or group of pixels from the ultrasound image data. The output layer may have neurons corresponding to locations of at least one of the anatomical structures depicted in the ultrasound image data. Each neuron of each layer may perform a processing function and pass the processed ultrasound image information to one of a plurality of neurons of a downstream layer for further processing. The processing performed by the segmentation processor 150 deep neural network (e.g., convolutional neural network) may identify the boundaries and locations of anatomical structures depicted in ultrasound image data with a high degree of probability.

The segmentation processor 150 may present identifications of the anatomical structures at the boundaries and locations superimposed on the first mode ultrasound image at the display system 134 and/or may store the identified anatomical structure boundaries and locations at archive 138 and/or any suitable data storage medium. In an exemplary embodiment, the identifications may be user-selectable to select the target anatomical structure. For example, an ultrasound operator may operate the user input device 130, provide a touch input on a touchscreen display system 134, or the like to select one of the plurality of anatomical structure identifications overlaid on the first mode ultrasound image. In a representative embodiment, each of the anatomical structures is associated with one or more pre-defined actions (e.g., acquisition of a first mode image according to pre-defined first mode imaging settings, acquisition of second mode image information according to second mode imaging settings, execution of one or more measurements, execution of image labeling according to a pre-defined standard format, etc.).

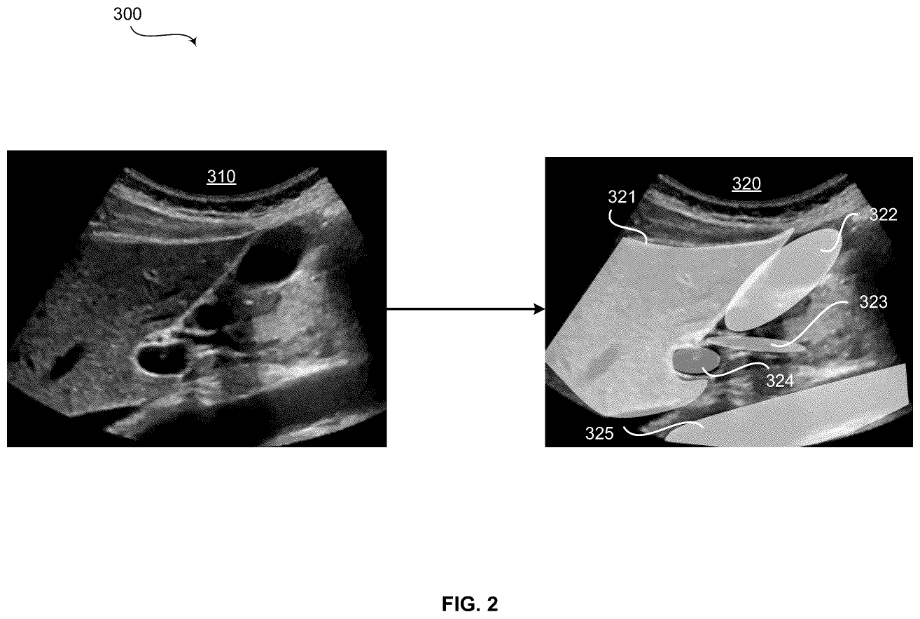

FIG. 2 illustrates screenshots 300 of an exemplary first mode ultrasound image 310 and an exemplary first mode ultrasound image 320 having identifications 321-325 of segmented anatomical structures, in accordance with various embodiments. Referring to FIG. 2, a first mode ultrasound image 310 and a first mode ultrasound image 320 having identifications 321-325 of segmented anatomical structures is shown. The first mode ultrasound images 310, 320 may be B-mode images or any suitable ultrasound images. In an exemplary embodiment, a first mode processor 140 processes an acquired and/or retrieved first mode ultrasound image dataset to generate ultrasound images 310 according to a first mode. The first mode image(s) 310 may be provided to the segmentation processor 150, which processes the first mode ultrasound image 310 to segment the anatomical structures depicted in the first mode ultrasound image 310. The segmentation processor 150 may be configured to cause a display system 134 to present identifications 321-325 of the boundaries and locations of each of the plurality of anatomical structures identified in the first mode ultrasound image 310. The identifications 321-325 may be superimposed on the first mode ultrasound image 310 resulting in the first module ultrasound image 320 having the identifications 321-325 of the anatomical structures. The identifications 321-325 may comprise shading, outlines, color-coding, markers, textual descriptions, and/or any suitable identifying information. The identifications 321-325 may be user-selectable by user input device 130, touchscreen, or the like to select a target anatomical structure. With reference to FIG. 2, the identified anatomical structures 321-325 may include a liver 321, gallbladder, 322, inferior vena cava (IVC) 323, common bile duct (CBD) 324, and aorta 325, for example. In various embodiments, the segmentation processor 150 may refrain from presenting the identifications 321-325 on the first mode ultrasound image 310, 320. The first mode ultrasound image 320 having the identifications 321-325 of the segmented anatomical structures may be presented at display system 134 and/or stored at archive 138 and/or any suitable data storage medium.

Referring again to FIG. 1, the segmentation processor 150 may comprise suitable logic, circuitry, interfaces and/or code that is operable to cause a display system 134 to present a pictogram corresponding to the first mode ultrasound image 310, 320. The pictogram may be an image, drawing, or the like that depicts at least the anatomical structures identified in the first mode ultrasound image 310, 320. The pictogram may be one of a plurality of pictograms stored at archive and/or any suitable data storage medium. The pictogram may be selectively retrieved and presented at the display system 134 based on the boundaries, locations, and/or identities of the anatomical structures segmented in the first mode ultrasound image 310, 320. The pictogram comprises selectable representations of anatomical structures corresponding with the segmented anatomical structures in the first mode ultrasound image 310, 320. The selection of one of the selectable representations may be a selection of the target anatomical structure. The pictogram may be displayed with, or instead of, the first mode ultrasound image 310, 320. In embodiments where the pictogram is presented with the first mode ultrasound image 310, 320, the segmentation processor 150 may be configured to depict the representations of the anatomical structures in the pictogram in a same manner as the identifications 321-325 in the first mode ultrasound image 310, 320. For example, corresponding anatomical structures may be color-coded, shaded, labeled, outlined, or the like to illustrate correspondence between the segmented identifications 321-325 in the first mode ultrasound image 310, 320 and the pictogram representations of the same anatomical structures. As an example, a segmented liver 321 may be shaded or outlined in red and a corresponding pictogram representation of a liver may also be identified by red shading, outlining, or the like. In various embodiments, the identifications 321-325 of the segmented anatomical structures in the first mode ultrasound image 310, 320 and/or the representations of the anatomical structures in the pictogram may be user-selectable to select the target anatomical structure. In a representative embodiment, each of the anatomical structures is associated with at least one pre-defined action (e.g., acquisition of a first mode image according to pre-defined first mode imaging settings, acquisition of second mode image information according to second mode imaging settings, execution of one or more measurements, execution of image labeling according to a pre-defined standard format, etc.).

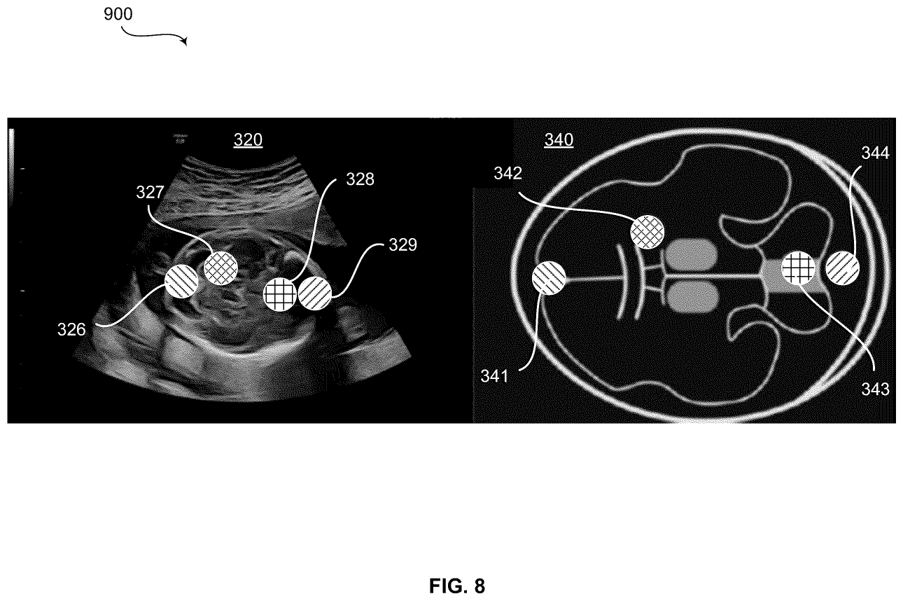

FIG. 8 illustrates a screenshot 900 of an exemplary first mode ultrasound image 320 having identifications 326-329 of segmented anatomical structures presented with a pictogram 340 having representations 341-344 of anatomical structures corresponding with the segmented anatomical structures in the first mode ultrasound image 320, in accordance with various embodiments. Referring to FIG. 8, a screenshot 900 is shown having a first mode ultrasound image 320 presented at a first display portion and a pictogram 340 presented at a second display portion adjacent the first display portion. The first mode ultrasound image 320 comprises identifications 326-329 of segmented anatomical structures depicted in the first mode ultrasound image 320. For example, the first mode ultrasound image 320 in FIG. 8 is a B-mode image of a fetal brain comprising a midline falx 326, cavum septum pellucidum 327, cerebellum 328, and cisterna magna 329. The pictogram 340 comprises representations 341-344 corresponding with the segmented anatomical structures depicted in the first mode ultrasound image 320. For example, the pictogram 340 in FIG. 8 is an illustration of a fetal brain comprising representations of a midline falx 341, cavum septum pellucidum 342, cerebellum 343, and cisterna magna 344. As shown in FIG. 8, each of the identifications 326-329 of the anatomical structures in the first mode ultrasound image 320 are shaded in a same manner as the corresponding representations of the anatomical structures provided in the pictogram 340. In an exemplary embodiment, the identifications 326-329 of the segmented anatomical structures in the first mode ultrasound image 320 and/or the corresponding representations 341-344 of the anatomical structures in the pictogram 340 may be user-selectable to select a target anatomical structure. In various embodiments, each of the anatomical structures is associated with one or more pre-defined actions.

Referring again to FIG. 1, the signal processor 132 may include a preset processor 160 that comprises suitable logic, circuitry, interfaces and/or code that may be operable to select and apply a preset of image settings that corresponds with a selected target anatomical structure during acquisition of first mode ultrasound images 310, 320. For example, presets of ultrasound imaging settings, such as gain, depth, frequency, line density/frame average (L/A), pulse repetition frequency (PRF), wall filter (WF), spatial filter/packet size (S/P), acoustic output, and/or any suitable imaging settings, may be stored in association with each of a plurality of anatomical structures. The presents of ultrasound imaging settings may be stored, for example, at archive 138 and/or any suitable data storage medium. The preset processor 160 may receive a selection of one of the plurality of anatomical structures depicted in the first mode ultrasound image 310, 320 via a user input device 130, touchscreen display 134, and/or any suitable user selection mechanism. For example, an ultrasound operator may navigate a pointer icon across a display device 134 via a mousing user input device 130, or provide a touch input on a touchscreen display 134, to select an identification 321-329 of a segmented anatomical structure in a first mode ultrasound image 310, 320 or a corresponding representation 341-344 of an anatomical structure in a pictogram 340, among other things. The preset processor 160 may receive the user selection and in response, may retrieve and apply the preset of ultrasound imaging settings specific to the selected anatomical structure in acquiring subsequent first mode ultrasound images 310, 320. The preset processor 160 may continue applying the preset of ultrasound imaging settings until a different target anatomical structure is selected, the ultrasound examination ends, or the like.

The signal processor 132 may include a second mode processor 170 that comprises suitable logic, circuitry, interfaces and/or code that may be operable to select and apply second mode imaging settings corresponding with a selected target anatomical structure for acquiring second ultrasound image information according to a second mode by the ultrasound probe 104. For example, the second mode may be a Color Flow mode, Power Doppler mode, B-Flow Color mode, shear wave elastography mode, or any suitable mode. The second mode may comprise a region of interest box that is placed in a first mode ultrasound image 310, 320. The placement of the region of interest box surrounding one of the plurality of anatomical structures 321-329 segmented in the first mode ultrasound image 310, 320 is a selection of the target anatomical structure in the first mode ultrasound image 310, 320. The selection of the target anatomical structure from the plurality of anatomical structures depicted in the first mode ultrasound image 310, 320 may be provided to the second mode processor 170. In a representative embodiment, each of a plurality of sets of second mode imaging settings may be associated with a different one of the plurality of anatomical structures. For example, different target anatomical structures may correspond with different second mode imaging settings due to the different hemodynamics of the different anatomical structures. As another example, the location of the target anatomical structure in the first mode ultrasound image may influence the selection of various second mode imaging settings, such as a depth of the target anatomical structure in the first mode ultrasound image influencing the selected second mode frequency setting, among other things. The second mode imaging settings may include, for example, gain, frequency, line density/frame average (L/A), pulse repetition frequency (PRF), wall filter (WF), spatial filter/packet size (S/P), acoustic output, and/or any suitable imaging settings for a second mode, such as Color Flow, Power Doppler, B-Flow Color, shear wave elastography, or the like. The sets of second mode imaging settings may be stored, for example, at archive 138 and/or any suitable data storage medium. The second mode processor 170 may receive the user selection of the placement of the region of interest box and in response, may retrieve and apply the second mode imaging settings specific to the selected anatomical structure in acquiring second mode ultrasound information. The second mode processor 170 may continue applying the set of second mode imaging settings until a different target anatomical structure is selected, the ultrasound examination ends, or the like. For example, a different target anatomical structure may be selected by an ultrasound operator moving the ultrasound probe 104 to position the different target anatomical structure within the region of interest box, by the ultrasound operator moving the region of interest box to the different target anatomical structure in the first mode ultrasound image 310, 320, or the like.

The ultrasound system 100 is configured to acquire second mode ultrasound information according to a second mode based on the second mode imaging settings selected by the second mode processor 170 in response to the target anatomical structure selection. The second mode processor 170 is configured to cause the display system 134 to present the region of interest box and the acquired second mode information within the region of interest box superimposed on the first mode ultrasound image 310, 320. The second mode processor 170 may be configured to update the second mode imaging settings in response to updated target anatomical structure selections. For example, the second mode processor 170 may receive an updated target anatomical structure selection if an ultrasound operator moves the ultrasound probe 104 to a different view to acquire ultrasound image data of a different target anatomical structure. As another example, the second mode processor 170 may receive an updated target anatomical structure selection if an ultrasound operator selects a different target anatomical structure within the same view, such as switching from a liver target structure 321 to an aorta 325 as a target structure in the abdominal view shown in FIG. 2. For example, the ultrasound operator may select the different target by navigating a cursor via a user input device 130 (e.g., mousing device, trackball, etc.) over the different target anatomical structure in the displayed first mode ultrasound image 310, 320 and providing a selection input (e.g., button depression), or providing a touch input of the different anatomical structure in the first mode ultrasound image 310, 320 presented at a touchscreen display 130, 134. As another example, the ultrasound operator may select a different target anatomical structure from a drop-down menu listing anatomical structures depicted in the current view.

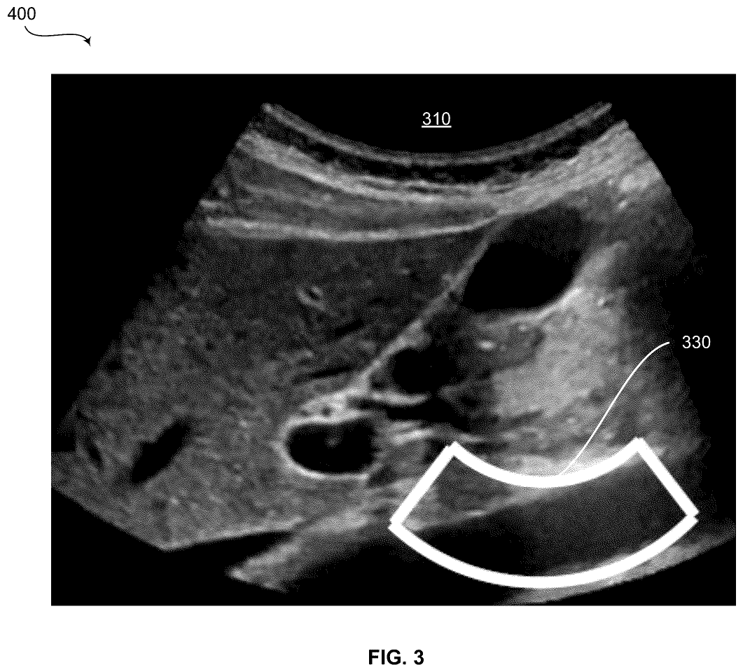

FIG. 3 illustrates a screenshot 400 of exemplary a region of interest box 330 placed on a first mode ultrasound image 310, in accordance with various embodiments. Referring to FIG. 3, a first mode ultrasound image 310 is shown with a region of interest box 330 placed over an anatomical structure. The region of interest box 330 may correspond with a second mode, such as a Color Flow mode, Power Doppler mode, B-Flow Color mode, shear wave elastography mode, or any suitable mode. The placement of the region of interest box 330 surrounding one of the plurality of anatomical structures depicted in the first mode ultrasound image 310 is a selection of the target anatomical structure in the first mode ultrasound image 310. In the case of FIG. 3, the abdominal aorta is selected based on the placement of the region of interest box 330. The selection of the target anatomical structure from the plurality of anatomical structures depicted in the first mode ultrasound image 310 may be provided to the second mode processor 170.

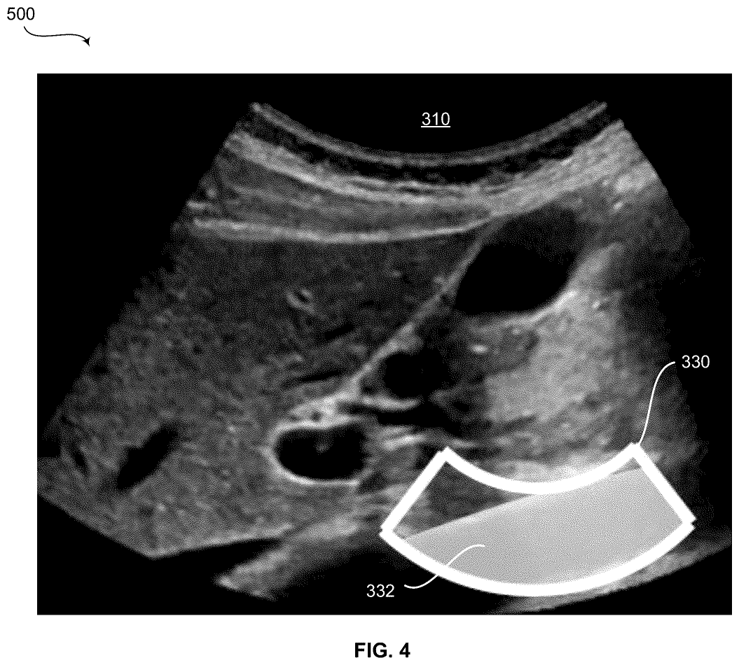

FIG. 4 illustrates a screenshot 500 of exemplary second mode ultrasound information 332 within a region of interest box 330 placed on a first mode ultrasound image 310, in accordance with various embodiments. Referring to FIG. 4, a first mode ultrasound image 310 is shown with a region of interest box 330 placed over an anatomical structure, and second mode ultrasound information 332 is presented within the region of interest box 330. The region of interest box 330 may correspond with a second mode, such as a Color Flow mode, Power Doppler mode, B-Flow Color mode, shear wave elastography mode, or any suitable mode. The placement of the region of interest box 330 surrounding one of the plurality of anatomical structures depicted in the first mode ultrasound image 310 is a selection of the target anatomical structure in the first mode ultrasound image 310. In the case of FIG. 4, the abdominal aorta is selected based on the placement of the region of interest box 330. The selection of the target anatomical structure from the plurality of anatomical structures depicted in the first mode ultrasound image 310 may be provided to the second mode processor 170, which selects and applies a set of second mode imaging settings corresponding with the abdominal aorta anatomical structure selected by placement of the region of interest box 330. The ultrasound system 100 is configured to acquire second mode ultrasound information 332 according to the second mode based on the second mode imaging settings selected by the second mode processor 170 in response to the target anatomical structure selection. The second mode processor 170 is configured to cause the display system 134 to present 500 the region of interest box 330 and the acquired second mode information 332 within the region of interest box 330 superimposed on the first mode ultrasound image 310.

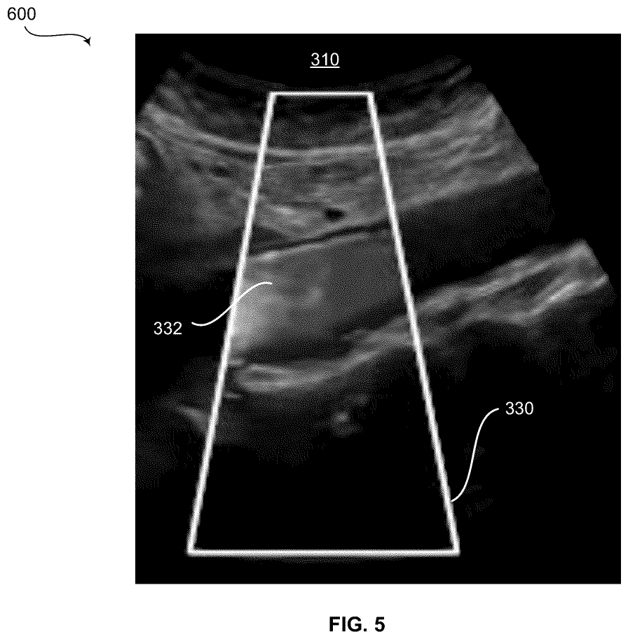

FIG. 5 illustrates a screenshot 600 of another exemplary second mode ultrasound information 332 within a region of interest box 330 placed on a first mode ultrasound image 310, in accordance with various embodiments. Referring to FIG. 5, the screenshot 600 includes a first mode ultrasound image 310 having a region of interest box 330 placed surrounding an abdominal aorta as the target anatomical structure. The region of interest box 330 is associated with a second mode, such as a Color Flow mode, Power Doppler mode, B-Flow Color mode, shear wave elastography mode, or any suitable mode. The ultrasound system 100 is configured to acquire second mode ultrasound information 332 according to the second mode based on second mode imaging settings associated with the abdominal aorta target anatomical structure selected based on the placement of the region of interest box 330. The second mode imaging settings include settings specifically configured for the abdominal aorta target anatomical structure, such as a scale of 30 centimeters per second, a gain of 10, and a low acoustic line density. The second mode processor 170 is configured to cause the display system 134 to present 600 the region of interest box 330 and the acquired second mode information 332 within the region of interest box 330 superimposed on the first mode ultrasound image 310.

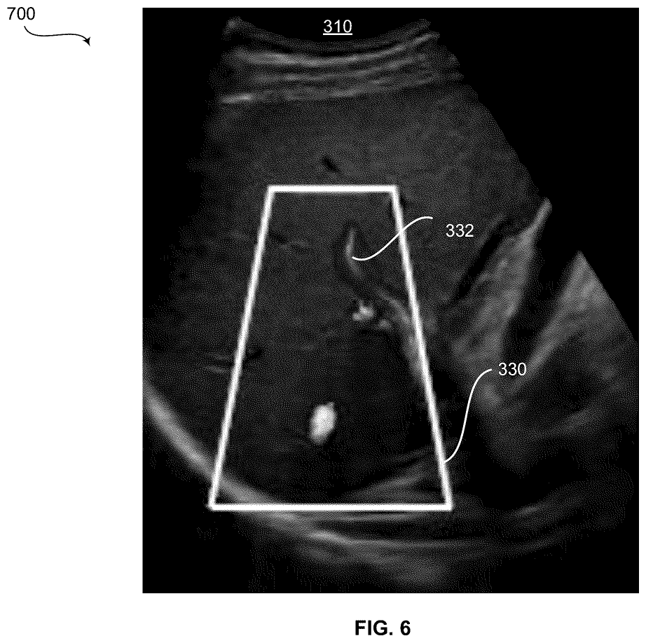

FIG. 6 illustrates a screenshot 700 of another exemplary second mode ultrasound information 332 within a region of interest box 330 placed on a first mode ultrasound image 310, in accordance with various embodiments. Referring to FIG. 6, the screenshot 700 includes a first mode ultrasound image 310 having a region of interest box 330 placed surrounding liver portal vein as the target anatomical structure. The region of interest box 330 is associated with a second mode, such as a Color Flow mode, Power Doppler mode, B-Flow Color mode, shear wave elastography mode, or any suitable mode. The ultrasound system 100 is configured to acquire second mode ultrasound information 332 according to the second mode based on second mode imaging settings associated with the liver portal vein target anatomical structure selected based on the placement of the region of interest box 330. The second mode imaging settings include settings specifically configured for the liver portal vein target anatomical structure, such as a scale of 20 centimeters per second, a gain of 15, and a mid acoustic line density. The second mode processor 170 is configured to cause the display system 134 to present 700 the region of interest box 330 and the acquired second mode information 332 within the region of interest box 330 superimposed on the first mode ultrasound image 310.

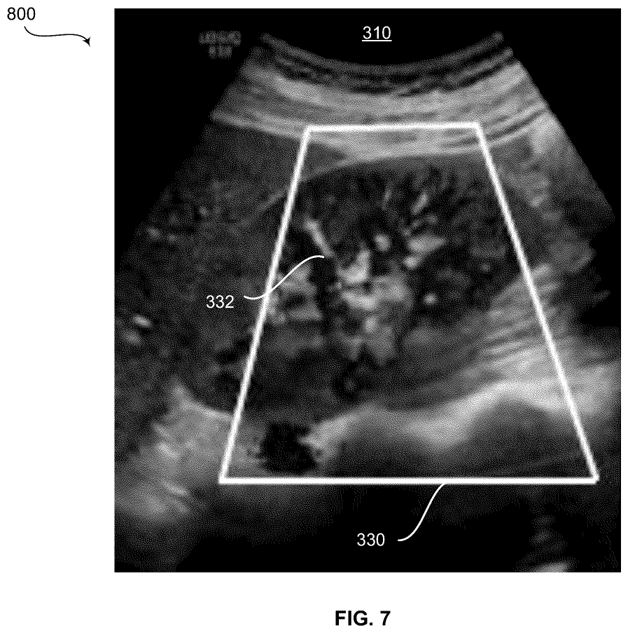

FIG. 7 illustrates a screenshot 800 of another exemplary second mode ultrasound information 332 within a region of interest box 330 placed on a first mode ultrasound image 310, in accordance with various embodiments. Referring to FIG. 7, the screenshot 800 includes a first mode ultrasound image 310 having a region of interest box 330 placed surrounding a kidney as the target anatomical structure. The region of interest box 330 is associated with a second mode, such as a Color Flow mode, Power Doppler mode, B-Flow Color mode, shear wave elastography mode, or any suitable mode. The ultrasound system 100 is configured to acquire second mode ultrasound information 332 according to the second mode based on second mode imaging settings associated with the kidney target anatomical structure selected based on the placement of the region of interest box 330. The second mode imaging settings include settings specifically configured for the kidney target anatomical structure, such as a scale of 10 centimeters per second, a gain of 20, and a high acoustic line density. The second mode processor 170 is configured to cause the display system 134 to present 800 the region of interest box 330 and the acquired second mode information 332 within the region of interest box 330 superimposed on the first mode ultrasound image 310.

Referring again to FIG. 1, the signal processor 132 may include measurement processor 180 that comprises suitable logic, circuitry, interfaces and/or code that may be operable to select and perform a measurement that corresponds with a target anatomical structure selected in the first mode ultrasound images 310, 320. For example, each of the different anatomical structures depicted in a first mode ultrasound image 310, 320 may be associated with one or more measurements. As an example, a kidney may be associated with a kidney length measurement, an abdominal aorta may be associated with an aorta vessel diameter measurement, a pancreas may be associated with a pancreas head size measurement, a gallbladder may be associated with a gallbladder wall thickness measurement, a spleen may be associated with a spleen size measurement, and a bladder may be associated with a bladder volume measurement, among other things. The measurements associated with each of the plurality of anatomical structures may be stored, for example, at archive 138 and/or any suitable data storage medium. The measurement processor 180 may receive a selection of one of the plurality of anatomical structures depicted in the first mode ultrasound image 310, 320 via a user input device 130, touchscreen display 134, and/or any suitable user selection mechanism. For example, an ultrasound operator may navigate a pointer icon across a display device 134 via a mousing user input device 130, or provide a touch input on a touchscreen display 134, to select an identification 321-329 of a segmented anatomical structure in a first mode ultrasound image 310, 320, or a corresponding representation 341-344 of an anatomical structure in a pictogram 340, among other things. The measurement processor 180 may receive the user selection and in response, may retrieve and perform the measurement specific to the selected anatomical structure in the first mode ultrasound image 310, 320. The measurement processor 180 may present the results of the performed measurement at the display system 134 and/or store the results of the performed measurement at archive 138 and/or any suitable data storage medium.

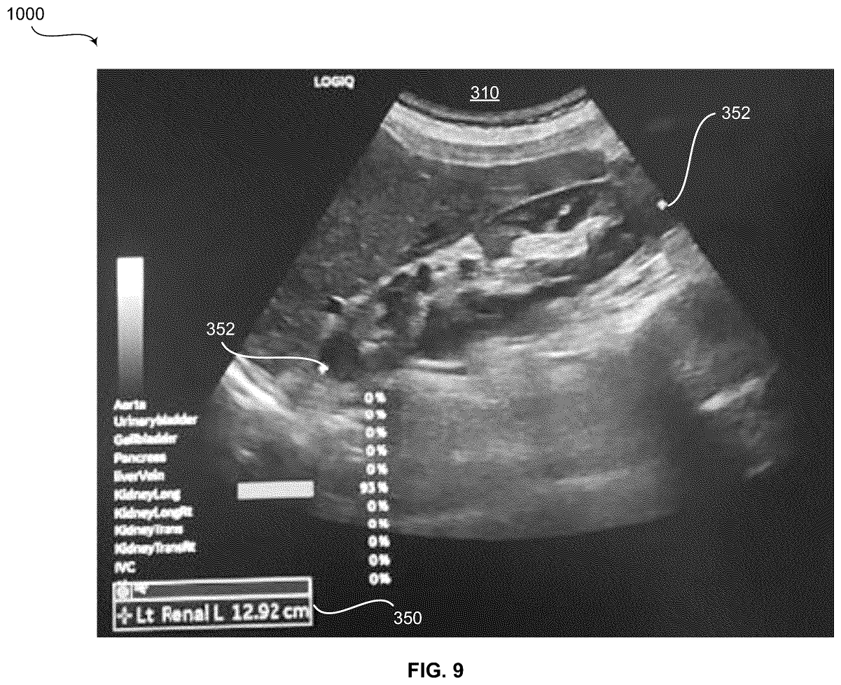

FIG. 9 illustrates a screenshot 1000 of an exemplary measurement 350 in a first mode ultrasound image 310, in accordance with various embodiments. Referring to FIG. 9, the screenshot 1000 illustrates a first mode ultrasound image 310 comprising a kidney as a target anatomical structure. The measurement 350 associated with the kidney target anatomical structure is a kidney long axis measurement 350. The first mode ultrasound image 310 is overlaid with measurement markers 352 provided at the automatically detected edges of the kidney and the measurement results 350 are presented in the screenshot 1000.

Referring again to FIG. 1, the signal processor 132 may include a labeling processor 190 that comprises suitable logic, circuitry, interfaces and/or code that may be operable to select and apply labels in a particular form that corresponds with a target anatomical structure selected in the first mode ultrasound images 310, 320. For example, each of the different anatomical structures depicted in a first mode ultrasound image 310, 320 may be associated with a standard labeling form. As an example, a gallbladder may be associated with a standard labeling form the dictates the gallbladder, pancreas, and kidney are identified in the first mode ultrasound image 310, 320. In various embodiments, the standard labeling form may dictate that the labels are full text or abbreviations. In certain embodiments, the labels may include outlining, color-coding, and/or any suitable labeling features. The standard labeling form associated with each of the plurality of anatomical structures may be stored, for example, at archive 138 and/or any suitable data storage medium. The labeling processor 190 may receive a selection of one of the plurality of anatomical structures depicted in the first mode ultrasound image 310, 320 via a user input device 130, touchscreen display 134, and/or any suitable user selection mechanism. For example, an ultrasound operator may navigate a pointer icon across a display device 134 via a mousing user input device 130, or provide a touch input on a touchscreen display 134, to select an identification 321-329 of a segmented anatomical structure in a first mode ultrasound image 310, 320, or a corresponding representation 341-344 of an anatomical structure in a pictogram 340, among other things. The labeling processor 190 may receive the user selection and in response, may retrieve and perform the standard labeling form specific to the selected anatomical structure in the first mode ultrasound image 310, 320. The labeling processor 190 may present the labels superimposed on the first mode ultrasound image 310, 320 at the display system 134 and/or store the labeled first mode ultrasound image 310, 320 at archive 138 and/or any suitable data storage medium.

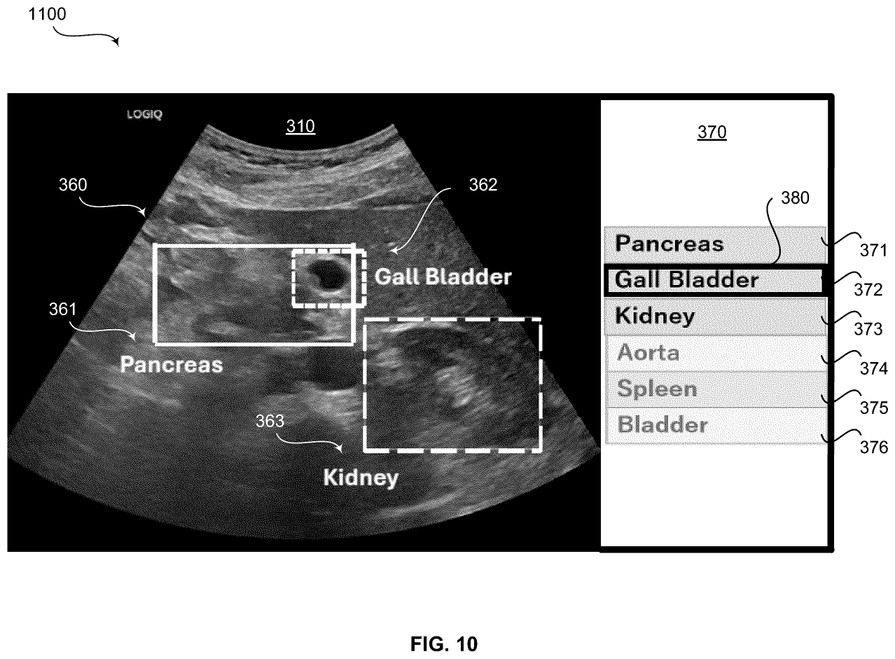

FIG. 10 illustrates a screenshot 1100 of an exemplary first mode ultrasound image 310 having a set of labels 360 corresponding with a selected anatomical structure, in accordance with various embodiments. Referring to FIG. 10, the screenshot 1100 illustrates an image display portion and an anatomical structure list 370. The anatomical structure list 370 comprises a list of anatomical structures 371-376, including at least the anatomical structures 371-373 present in the first mode ultrasound image 310. In certain embodiments, other nearby anatomical structures 374-376 may also be listed but may be grayed out. The list 370 of anatomical structures 371-376 may include a target identifier 380, such as highlighting, bolding, outlining, coloring, or the like. The target identifier 380 may identify the selected target anatomical structure. In various embodiments, an ultrasound operator may update the selected anatomical structure by selecting another of the anatomical structures 371-376 in the anatomical structure list 370, such as using a touch input or a user input device 130. In an exemplary embodiment, the anatomical structures 371-376 in the list may be color-coded or otherwise identified in a manner to match corresponding anatomical structures labeled 361-363 in the first mode ultrasound image 310. In certain embodiments, the anatomical structure list 370 may be omitted. The image display portion comprises a first mode ultrasound image 310 having labels 360, including a pancreas label 361, a gallbladder label 362, and a kidney label 363. The labels 360 may include a textual portion and/or a marker surrounding the anatomical structure. In various embodiments, the marker may be an icon, symbol, or any suitable marker. The marker may be color-coded, shaded, dashed, solid, or the like. In certain embodiments, the marker may be omitted. The textual portion may be full text, abbreviations, or the like. In an exemplary embodiment, the textual portion may be color-coded. In a representative embodiment, the color-coding or other identification of the labels 361-363 may match color-coding or other identification of the corresponding listed anatomical structures 371-373. The anatomical structures that are labeled and the manner in which the anatomical structures are labeled is based on a standard labeling form that corresponds with a selected target anatomical structure. Referring to FIG. 10, the target anatomical structure is the gallbladder, and the anatomical structures that are labeled 361-363 along with the manner in which the labeling 360 appears is based on a standard labeling form for the gallbladder. The anatomical structures that are labeled and the standard labeling form may be different for other anatomical structures that may be selected as the target anatomical structure.

The display system 134 may be any device capable of communicating visual information to a user. For example, a display system 134 may include a liquid crystal display, a light emitting diode display, and/or any suitable display or displays. The display system 134 can be operable to present first mode ultrasound images 310, 320, identifications 321-329 of segmented anatomical structures, region of interest boxes 330, second mode ultrasound information 332, pictograms 340, representations 341-344 of anatomical structures in pictograms 340, measurement results 350, labeling 360, anatomical structure lists 370, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores first mode ultrasound images 310, 320, identifications 321-329 of segmented anatomical structures, instructions for segmenting first mod ultrasound images 310, 320, presets of first mode imaging settings associated with particular anatomical structures, region of interest boxes 330, second mode imaging settings associated with particular anatomical structures, second mode ultrasound information 332, pictograms 340, representations 341-344 of anatomical structures in pictograms 340, measurement 350 associated with particular anatomical structures, standard labeling forms 360 associated with particular anatomical structures, and/or anatomical structure lists 370, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural networks (e.g., artificial intelligence model(s)) inferenced (i.e., deployed) by the signal processor 132 and/or segmentation processor 150. For example, the training engine 210 may apply classified anatomical structures to train the object segmentation networks inferenced by the segmentation processor 150 to automatically segment anatomical structures 321-329 in first mode ultrasound images 310, 320. The classified anatomical structures may include an input image and a ground truth binary image (i.e., mask) of the manually segmented anatomical structures 321-329. The training engine 210 may be configured to optimize the object segmentation networks by adjusting the weighting of the object segmentation networks to minimize a loss function between the input ground truth mask and an output predicted mask.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

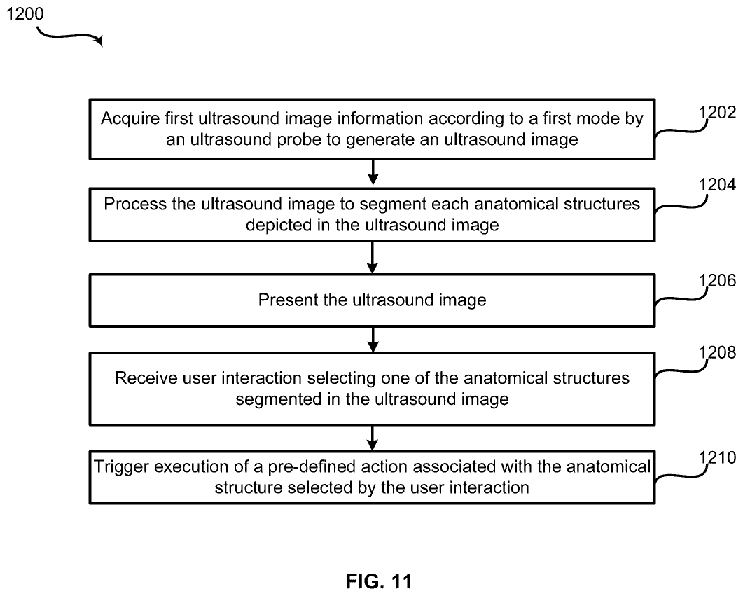

FIG. 11 is a flow chart 1200 illustrating exemplary steps 1202-1210 that may be utilized for automatically executing pre-defined actions based on anatomical structure segmentation and selection during ultrasound imaging, in accordance with various embodiments. Referring to FIG. 11, there is shown a flow chart 1200 comprising exemplary steps 1202 through 1210. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 1202, an ultrasound probe 104 of an ultrasound system 100 may acquire a first ultrasound image information according to a first mode to generate a first mode ultrasound image 310, 320. For example, an ultrasound probe 104 in the ultrasound system 100 may be operable to perform an ultrasound scan of a region of interest, such as an abdominal region. The ultrasound scan may be performed according to the first mode, such as a B-mode or any suitable image acquisition mode. The first ultrasound image dataset may be received by the first mode processor 140 of the signal processor 132 and/or stored to archive 138 or any suitable data storage medium from which the first mode processor 140 may retrieve the first ultrasound image information. The first mode processor 140 of the signal processor 132 of the ultrasound system 100 may be configured to process the acquired and/or retrieved first mode ultrasound image information to generate ultrasound images 310, 320 according to the first mode. As an example, the first mode may be a B-mode and the first mode processor 140 may be configured to process received first mode ultrasound image information into B-mode image(s) 310, 320.

At step 1204, the signal processor 132 of the ultrasound system 100 may process the first mode ultrasound image 310, 320 to segment each anatomical structure 321-329 depicted in the first mode ultrasound image 310, 320. For example, a segmentation processor 150 of the signal processor 132 of the ultrasound system 100 may be configured to determine boundaries and locations of anatomical structure 321-329 depicted in the first mode ultrasound image 310, 320. The segmentation processor 150 may determine locations of, for example, one or more of a liver, kidney, gallbladder, aorta, pancreas, spleen, and/or inferior vena cava, among other anatomical structures, depicted in a first mode ultrasound image 310, 320 of an abdomen. In various embodiments, the segmentation processor 150 inferences an object segmentation deep learning model, or any suitable image analysis algorithms, to identify boundaries and locations of anatomical structures depicted in the first mode ultrasound image 310, 320.

At step 1206, the signal processor 132 of the ultrasound system 100 may cause a display system 134 of the ultrasound system 100 to present the first mode ultrasound image 310, 320. For example, the first mode processor 140 of the signal processor 132 may cause the display system 134 to present the first mode ultrasound image 310, 320. The first mode ultrasound image 310, 320 may be the first mode ultrasound image 310 generated at step 1202 or the first mode ultrasound image 320 having identifications 321-329 of the anatomical structures segmented at step 1204 superimposed by the segmentation processor 150 on the first mode ultrasound image 320. In various embodiments, the segmentation processor 150 may cause a display system 134 to present a pictogram 340 corresponding to the first mode ultrasound image 310, 320. The pictogram 340 may be an image, drawing, or the like that depicts at least the anatomical structures identified in the first mode ultrasound image 310, 320. The pictogram 340 may be one of a plurality of pictograms 340 stored at archive 138 and/or any suitable data storage medium. The pictogram 340 may be selectively retrieved and presented at the display system 134 based on the boundaries, locations, and/or identities of the anatomical structures segmented in the first mode ultrasound image 310, 320. The pictogram 340 comprises selectable representations 341-344 of anatomical structures corresponding with the segmented anatomical structures 321-329 in the first mode ultrasound image 310, 320. In embodiments where the pictogram 340 is presented with the first mode ultrasound image 310, 320, the segmentation processor 150 may be configured to depict the representations 341-344 of the anatomical structures in the pictogram 340 in a same manner as the identifications 326-329 in the first mode ultrasound image 310, 320. For example, corresponding anatomical structures may be color-coded, shaded, labeled, outlined, or the like to illustrate correspondence between the segmented identifications 326-329 in the first mode ultrasound image 310, 320 and the pictogram 340 representations 341-344 of the same anatomical structures.

At step 1208, the signal processor 132 of the ultrasound system 100 may receive a user interaction selecting one of the plurality of anatomical structures 321-329 segmented in the first mode ultrasound image 310, 320. For example, the identifications 321-329 of the segmented anatomical structures in the first mode ultrasound image 310, 320 and/or the representations 341-344 of the anatomical structures in the pictogram 340 may be user-selectable to select the target anatomical structure. As another example, the placement of a region of interest box 330 surrounding one of the anatomical structures segmented in the first mode ultrasound image 310, 320 may be a selection of the target anatomical structure. The region of interest box 330 may be associated with a second mode, different from the first mode. For example, the first mode may be a 2D mode (e.g., B-mode, biplane mode, triplane mode, or the like) and the second mode may be a Color Flow mode, Power Doppler mode, B-Flow Color mode, shear wave elastography mode, or any suitable mode. In various embodiments, if a user places the region of interest box 330 surrounding more than one of the anatomical structures, the at least one processor 132 may be configured to select the one of the plurality of anatomical structures 321-329 based on user-defined or default preferences. For example, the user or default preferences may define that the signal processor 132 selects the anatomical structure closest to the center of the region of interest box 330. As another example, the user or default preferences may define that the signal processor 132 selects the anatomical structure having a highest confidence score.

At step 1210, the signal processor 132 of the ultrasound system 100 may trigger execution of a pre-defined action associated with the anatomical structure selected by the user interaction. For example, each of the anatomical structures segmented in the first mode ultrasound image 310, 320 and selectable by a user interaction (e.g., selectable identification 321-329 of the segmented anatomical structures in the first mode ultrasound image 310, 320, selectable representation 341-344 of the segmented anatomical structures in the pictogram 340, selectable anatomical structure corresponding to placement of a region of interest box 330, etc.) is associated with at least one pre-defined action. In an exemplary embodiment, the pre-defined action may be the acquisition of a subsequent first mode ultrasound image 310, 320 according to a pre-defined preset of first mode imaging settings associated with the selected target anatomical structure. In a representative embodiment, the pre-defined action may be the acquisition of second mode image information 332 within a region of interest box 330 according to second mode imaging settings associated with the selected target anatomical structure. In various embodiments, the pre-defined action may be execution of one or more measurements 350 of the selected target anatomical structure. In certain embodiments, the pre-defined action may be execution of image labeling 360 according to a pre-defined standard format associated with the selected target anatomical structure.

Aspects of the present disclosure provide a method 1200 and system 100 for automatically executing pre-defined actions based on anatomical structure segmentation and selection during ultrasound imaging. In accordance with various embodiments, the method 1200 may comprise acquiring 1202, by an ultrasound probe 104 of an ultrasound system 100, first ultrasound image information according to a first mode. The first ultrasound image information comprises a first mode ultrasound image 310, 320 depicting a plurality of anatomical structures 321-329. The method 1200 may comprise processing 1204, by at least one processor 132, 150 of the ultrasound system 100, the first mode ultrasound image 310 to segment each of the plurality of anatomical structures 321-329 depicted in the first mode ultrasound image 310. The method 1200 may comprise causing 1206, by the at least one processor 132, 140, a display system 134 to present the first mode ultrasound image 310, 320. The method 1200 may comprise receiving 1208, by the at least one processor 132, 160, 170, 180, 190, a user interaction selecting one of the plurality of anatomical structures 321-329 segmented in the first mode ultrasound image 310, 320. The method 1200 may comprise triggering 1210, by the at least one processor 132, 160, 170, 180, 190 in response to the user interaction, execution of a pre-defined action associated with the one of the plurality of anatomical structures 321-329 selected by the user interaction.

In an exemplary embodiment, the method 1200 may comprise causing 1206, by the at least one processor 132, 140, 150, the display system 134 to present an identification 321-329 of each of the plurality of anatomical structures segmented in the first mode ultrasound image 320. The user interaction may be a selection of one of the identifications 321-329 in the first mode ultrasound image 320. In a representative embodiment, the user interaction is placement of a region of interest box 330 over the one of the plurality of anatomical structures. The region of interest box 330 may be associated with a second mode, different from the first mode. The placement of the region of interest box 330 may trigger acquisition of second ultrasound image information 332 according to the second mode at the location of the placement. In various embodiments, the method 1200 may comprise selecting 1210, by the at least one processor 132, 170, second mode imaging settings based on the one of the plurality of anatomical structures within the region of interest box 330. The second ultrasound image information 332 may be acquired based on the second mode imaging settings. The first mode may be a B-mode. The second mode may be one of a Color Flow mode, Power Doppler mode, B-Flow Color mode, or shear wave elastography mode. In certain embodiments, the method 1200 may comprise causing 1206, by the at least one processor 132, 150, the display system 134 to present a pictogram 340 comprising a representation 341-344 of each of the plurality of anatomical structures 326-329 depicted in the first mode ultrasound image 310, 320. In an exemplary embodiment, each of the of the plurality of anatomical structures 326-329 segmented in the first mode ultrasound image 310, 320 is identified by a different color. Each representation 341-344 of each of the plurality of anatomical structures in the pictogram 340 may be identified by a corresponding one of the different color. In a representative embodiment, the user interaction is a selection of one of the representations 341-344 in the pictogram 340. In various embodiments, the pre-defined action is acquisition of a B-mode image 310, 320 according to first mode imaging settings associated with the one of the plurality of anatomical structures selected by the user interaction. In certain embodiments, the pre-defined action is an automated measurement 350 associated with the one of the plurality of anatomical structures selected by the user interaction. In an exemplary embodiment, the pre-defined action is labeling 360 of the first mode ultrasound image 310 and/or another image according to a standard labeling form associated with the one of the plurality of anatomical structures selected by the user interaction.

Various embodiments provide a system 100 for automatically executing pre-defined actions based on anatomical structure segmentation and selection during ultrasound imaging. The ultrasound system 100 may comprise an ultrasound probe 104, a display system 134, and at least one processor 132, 140, 150, 160, 170, 180, 190. The ultrasound probe 104 may be operable to acquire first ultrasound image information according to a first mode. The first ultrasound image information may comprise a first mode ultrasound image 310, 320 depicting a plurality of anatomical structures 321-329. The at least one processor 132, 140 may be configured to process the first mode ultrasound image 310, 320 to segment each of the plurality of anatomical structures 321-329 depicted in the first mode ultrasound image 310, 320. The at least one processor 132, 140 may be configured to cause a display system 134 to present the first mode ultrasound image 310, 320. The at least one processor 132, 160, 170, 180, 190 may be configured to receive a user interaction selecting one of the plurality of anatomical structures 321-329 segmented in the first mode ultrasound image 310, 320. The at least one processor 132, 160, 170, 180, 190 may be configured to trigger execution of a pre-defined action associated with the one of the plurality of anatomical structures 321-329 selected by the user interaction in response to receiving the user interaction. The display system 134 may be configured to present the first mode ultrasound image 310, 320.

In a representative embodiment, an identification 321-329 of each of the plurality of anatomical structures segmented in the first mode ultrasound image 310, 320 is presented in the first mode ultrasound image 320 at the display system 134. The user interaction is a selection of one of the identifications 321-329 in the first mode ultrasound image 320. In various embodiments, the user interaction is placement of a region of interest box 330 over the one of the plurality of anatomical structures 321-329. The region of interest box 330 may be associated with a second mode, different from the first mode. The placement of the region of interest box 330 may trigger acquisition of second ultrasound image information 332 according to the second mode at the location of the placement. The first mode may be a B-mode. The second mode may be one of a Color Flow mode, Power Doppler mode, B-Flow Color mode, or shear wave elastography mode. In certain embodiments, the at least one processor 132, 150 is configured to cause the display system 134 to present a pictogram 340 comprising a representation 341-344 of each of the plurality of anatomical structures 321-329 depicted in the first mode ultrasound image 310, 320. In an exemplary embodiment, each of the of the plurality of anatomical structures 321-329 segmented in the first mode ultrasound image 310, 320 is identified by a different color. Each representation 341-344 of each of the plurality of anatomical structures in the pictogram 340 is identified by a corresponding one of the different color. In a representative embodiment, the user interaction is a selection of one of the representations 341-344 in the pictogram 340. In various embodiments, the pre-defined action is acquisition of a B-mode image 310, 320 according to first mode imaging settings associated with the one of the plurality of anatomical structures 321-329 selected by the user interaction. In certain embodiments, the pre-defined action is an automated measurement 350 associated with the one of the plurality of anatomical structures 321-329 selected by the user interaction. In an exemplary embodiment, the pre-defined action is a labeling 360 of the first mode ultrasound image 310, 320 and/or another image according to a standard labeling form associated with the one of the plurality of anatomical structures 321-329 selected by the user interaction.

Certain embodiments provide a system 100 for automatically executing pre-defined actions based on anatomical structure segmentation and selection during ultrasound imaging. The ultrasound system 100 may comprise an ultrasound probe 104, a display system 134, and at least one processor 132, 140, 150, 160, 170, 180, 190. The ultrasound probe 104 may be operable to acquire first ultrasound image information according to a first mode. The first ultrasound image information may comprise a first mode ultrasound image 310, 320 depicting a plurality of anatomical structures 321-329. The first mode may be a B-mode. The ultrasound probe 104 may be operable to acquire second ultrasound image information 332 according to a second mode based on a region of interest box 330. The second ultrasound image information 332 may be acquired based on second mode imaging settings. The second mode may be one of a Color Flow mode, Power Doppler mode, B-Flow Color mode, or shear wave elastography mode. The at least one processor 132, 150 may be configured to process the first mode ultrasound image 310, 320 to segment each of the plurality of anatomical structures 321-329 depicted in the first mode ultrasound image 310, 320. The at least one processor 132, 140 may be configured to cause a display system 134 to present the first mode ultrasound image 310, 320. The at least one processor 132, 160, 170, 180, 190 may be configured to receive a user interaction selecting one of the plurality of anatomical structures 321-329 segmented in the first mode ultrasound image 310, 320. The user interaction may be placement of a region of interest box 330 over the one of the plurality of anatomical structures. The region of interest box 330 may be associated with the second mode. The at least one processor 132, 170 may be configured to trigger acquisition of the second ultrasound image information 132 by the ultrasound probe 104 at the location of the placement of the region of interest box 330 and in response to receiving the placement of the region of interest box 330. The at least one processor 132, 170 may select the second mode imaging settings based on the one of the plurality of anatomical structures 321-329 within the region of interest box 330. The at least one processor 132, 140, 170 may be configured to cause a display system 134 to present the second ultrasound image information 332 with the region of interest box 330 placed over the one of the plurality of anatomical structures 321-329 selected by the user interaction of the placement of the region of interest box 330. The display system 134 may be configured to present the first mode ultrasound image 310, 320. The display system 134 may be configured to present the second ultrasound image information 332 with the region of interest box 330 placed on the first mode ultrasound image 310, 320.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for automatically executing pre-defined actions based on anatomical structure segmentation and selection during ultrasound imaging.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method, comprising:
acquiring, by an ultrasound probe of an ultrasound system, first ultrasound image information according to a first mode, wherein the first ultrasound image information comprises a first mode ultrasound image depicting a plurality of anatomical structures;
processing, by at least one processor of the ultrasound system, the first mode ultrasound image to segment each of the plurality of anatomical structures depicted in the first mode ultrasound image;
causing, by the at least one processor, a display system to present the first mode ultrasound image;
receiving, by the at least one processor, a user interaction selecting one of the plurality of anatomical structures segmented in the first mode ultrasound image; and
triggering, by the at least one processor in response to the user interaction, execution of a pre-defined action associated with the one of the plurality of anatomical structures selected by the user interaction.

2. The method of claim 1, comprising causing, by the at least one processor, the display system to present an identification of each of the plurality of anatomical structures segmented in the first mode ultrasound image, wherein the user interaction is a selection of one of the identification in the first mode ultrasound image.

3. The method of claim 1, wherein:
the user interaction is placement of a region of interest box over the one of the plurality of anatomical structures, the region of interest box associated with a second mode, different from the first mode; and
the placement of the region of interest box triggers acquisition of second ultrasound image information according to the second mode at the location of the placement.

4. The method of claim 3, comprising selecting, by the at least one processor, second mode imaging settings based on the one of the plurality of anatomical structures within the region of interest box, wherein:
the second ultrasound image information is acquired based on the second mode imaging settings;
the first mode is B-mode; and
the second mode is one of a Color Flow mode, Power Doppler mode, B-Flow Color mode, or shear wave elastography mode.

5. The method of claim 1, comprising causing, by the at least one processor, the display system to present a pictogram comprising a representation of each of the plurality of anatomical structures depicted in the first mode ultrasound image.

6. The method of claim 5, wherein:
each of the of the plurality of anatomical structures segmented in the first mode ultrasound image is identified by a different color; and
each representation of each of the plurality of anatomical structures in the pictogram is identified by a corresponding one of the different color.

7. The method of claim 5, wherein the user interaction is a selection of one of the representation in the pictogram.

8. The method of claim 1, wherein the pre-defined action is acquisition of a B-mode image according to first mode imaging settings associated with the one of the plurality of anatomical structures selected by the user interaction.

9. The method of claim 1, wherein the pre-defined action is an automated measurement associated with the one of the plurality of anatomical structures selected by the user interaction.

10. The method of claim 1, wherein the pre-defined action is labeling of the first mode ultrasound image and/or another image according to a standard labeling form associated with the one of the plurality of anatomical structures selected by the user interaction.

11. An ultrasound system, comprising:
an ultrasound probe operable to acquire first ultrasound image information according to a first mode, wherein the first ultrasound image information comprises a first mode ultrasound image depicting a plurality of anatomical structures;
at least one processor configured to:
process the first mode ultrasound image to segment each of the plurality of anatomical structures depicted in the first mode ultrasound image;
cause a display system to present the first mode ultrasound image;
receive a user interaction selecting one of the plurality of anatomical structures segmented in the first mode ultrasound image; and
trigger execution of a pre-defined action associated with the one of the plurality of anatomical structures selected by the user interaction in response to receiving the user interaction; and
the display system configured to present the first mode ultrasound image.

12. The ultrasound system of claim 11, wherein:
an identification of each of the plurality of anatomical structures segmented in the first mode ultrasound image is presented in the first mode ultrasound image at the display system; and
the user interaction is a selection of one of the identification in the first mode ultrasound image.

13. The ultrasound system of claim 11, wherein:
the user interaction is placement of a region of interest box over the one of the plurality of anatomical structures, the region of interest box associated with a second mode, different from the first mode;
the placement of the region of interest box triggers acquisition of second ultrasound image information according to the second mode at the location of the placement;
the first mode is B-mode; and
the second mode is one of a Color Flow mode, Power Doppler mode, B-Flow Color mode, or shear wave elastography mode.

14. The ultrasound system of claim 11, wherein the at least one processor is configured to cause the display system to present a pictogram comprising a representation of each of the plurality of anatomical structures depicted in the first mode ultrasound image.

15. The ultrasound system of claim 14, wherein:
each of the of the plurality of anatomical structures segmented in the first mode ultrasound image is identified by a different color; and
each representation of each of the plurality of anatomical structures in the pictogram is identified by a corresponding one of the different color.

16. The ultrasound system of claim 14, wherein the user interaction is a selection of one of the representation in the pictogram.

17. The ultrasound system of claim 11, wherein the pre-defined action is acquisition of a B-mode image according to first mode imaging settings associated with the one of the plurality of anatomical structures selected by the user interaction.

18. The ultrasound system of claim 11, wherein the pre-defined action is an automated measurement associated with the one of the plurality of anatomical structures selected by the user interaction.

19. The ultrasound system of claim 11, wherein the pre-defined action is a labeling of the first mode ultrasound image and/or another image according to a standard labeling form associated with the one of the plurality of anatomical structures selected by the user interaction.

20. An ultrasound system, comprising:
an ultrasound probe operable to:
acquire first ultrasound image information according to a first mode, wherein the first ultrasound image information comprises a first mode ultrasound image depicting a plurality of anatomical structures, and wherein the first mode is B-mode; and
acquire second ultrasound image information according to a second mode based on a region of interest box, wherein the second ultrasound image information is acquired based on second mode imaging settings, and wherein the second mode is one of a Color Flow mode, Power Doppler mode, B-Flow Color mode, or shear wave elastography mode;
at least one processor configured to:
process the first mode ultrasound image to segment each of the plurality of anatomical structures depicted in the first mode ultrasound image;
cause a display system to present the first mode ultrasound image;
receive a user interaction selecting one of the plurality of anatomical structures segmented in the first mode ultrasound image, wherein the user interaction is placement of a region of interest box over the one of the plurality of anatomical structures, the region of interest box associated with the second mode;
trigger acquisition of the second ultrasound image information by the ultrasound probe at the location of the placement of the region of interest box and in response to receiving the placement of the region of interest box, wherein the at least one processor selects the second mode imaging settings based on the one of the plurality of anatomical structures within the region of interest box; and
cause a display system to present the second ultrasound image information with the region of interest box placed over the one of the plurality of anatomical structures selected by the user interaction of the placement of the region of interest box; and
the display system configured to present:
the first mode ultrasound image; and
the second ultrasound image information with the region of interest box placed on the first mode ultrasound image.
